(19) United States
(12) Patent Application Publication
(10) Pub. No.: US 2024/0351011 A1
(43) Pub. Date: Oct. 24, 2024

(54) CATALYST COMPOSITION, ITS PREPARATION AND USE

(71) Applicant: SHELL USA, INC., Houston, TX (US)

(72) Inventors: Roy VAN DEN BERG, Amsterdam (NL); Erik ZUIDEMA, Amsterdam (NL); Patrick VANDER HOOGERSTRAETE, Wondelgem (BE); Wiebe KIJLSTRA, Amsterdam (NL); Milos ROOSE, Wondelgem (BE)

(21) Appl. No.: 18/686,909

(22) PCT Filed: Sep. 7, 2022
(86) PCT No.: PCT/EP2022/074867

§ 371 Date: Feb. 27, 2024

(30) Foreign Application Priority Data
Sep. 13, 2021 (EP) 21196411.9

(51) Int. Cl. B01J 29/44; B01J 6/00; B01J 21/06; B01J 37/00; B01J 37/02; C07C 5/27

(52) U.S. Cl. CPC B01J 29/44; B01J 6/001; B01J 21/06; B01J 37/0018; B01J 37/0207; B01J 37/0209; B01J 37/0213; C07C 5/2775; B01J 2231/52; C07C 2529/44

(57) ABSTRACT

A process for the preparation of a catalyst composition, which process comprises: forming a carrier from a mixture comprising a pentasil zeolite, one or more porous refractory oxide binders selected from alumina, amorphous silica-alumina, aluminum phosphate, magnesia, chromia, titania, boria and silica, and an aqueous solution of a zirconia precursor, and impregnating said carrier with metal dopants comprising one or more Group 10 metals selected from platinum, palladium and mixtures thereof in a total amount in the range of from 0.001 to 1 wt. % and, optionally, in the range of from 0.01 to 0.5 wt. % tin, based on the total weight of the catalyst composition; a catalyst composition prepared by said process; and a process for the use of said catalyst composition in xylene isomerisation are provided.

### FIELD OF THE INVENTION

The present invention relates to a catalyst composition, its preparation, and its use in xylene isomerisation.

### BACKGROUND OF THE INVENTION

Ethylbenzene (EB) is one of the aromatic hydrocarbons that is obtained from naphtha pyrolysis or reformate. Reformate is an aromatic product obtained by the catalysed conversion of straight-run hydrocarbons boiling in the 70 to 190° C. range, such as straight-run naphtha. The catalysts used for the production of reformate are often platinum-on-alumina catalysts. On conversion to reformate, the aromatics content is considerably increased, and the resulting hydrocarbon mixture becomes highly desirable as a source of valuable chemical intermediates and as a component for gasoline. The principal components are a group of aromatics often referred to as BTX: benzene, toluene, and the xylenes, including ethylbenzene. Other components may be present such as their hydrogenated homologues, e.g. cyclohexane.

Of the BTX group, the most valuable components are benzene and the xylenes, and therefore BTX is often subjected to processing to increase the proportion of those two aromatics: hydrodealkylation of toluene to benzene and toluene disproportionation to benzene and xylenes. Within the xylenes, para-xylene is the most useful commodity and xylene isomerisation or transalkylation processes have been developed to increase the proportion of para-xylene. A further process that can be applied is the hydrodealkylation of ethylbenzene to benzene.

Generally, it is preferred to isolate BTX from the reformate stream, then isolate the C₈ aromatics by distillation, followed by extraction of para-xylene via selective adsorption or crystallization. The para-xylene lean C₈ aromatics stream is then subjected to xylene isomerisation with the aim of maximising the para-xylene component to be able to recycle the stream and extract more para-xylene. This process is the subject of the present invention. To avoid build-up of ethylbenzene in the recycle stream, ethylbenzene often has to be converted as well. Typically, this is done either by dealkylating ethylbenzene to generate valuable benzene or by reforming ethylbenzene to xylenes to increase the yield of xylenes. In practice, often catalyst systems are used to isomerize the xylenes to equilibrium and simultaneously either reform ethylbenzene to xylenes or dealkylate ethylbenzene.

EP 0018498 A1 is concerned with catalysts suitable for xylene isomerisation and the simultaneous dealkylation of ethylbenzene and reviews a number of earlier proposals for the use of platinum ZSM-series zeolitic catalysts. Generally, such catalysts are shown to have a superior activity in isomerising xylenes and to dealkylate ethylbenzene but are required to be used at high temperatures as there is a tendency for platinum to hydrogenate the benzene ring and to cause other undesirable side-reactions such as disproportionation and transalkylation at the low temperatures that are preferred for xylene isomerisation. The proposal of EP 0018498 A1 is to use a second metal, which is preferably tin, barium, titanium, indium and cadmium, in combination with platinum and a high-silica zeolite bound with a refractory inorganic oxide, which in all of the examples is alumina.

EP 0425712 A1 describes an improved catalyst for simultaneous xylene isomerisation and ethylbenzene dealkylation, which is formed by combining a group VIII metal, preferably platinum, with a lead component, and a halogen component, on a carrier of a pentasil zeolite and an inorganic oxide binder, preferably alumina, such that a specific ratio of lead to Group VIII metal is achieved and such that the bulk of the Group VIII and lead components are combined with the binder material.

WO 2009/016143 A1 describes a catalyst composition comprising a) a carrier which comprises at least 30 wt. % of a binder selected from silica, zirconia and titania; at least 20 wt. % of a pentasil zeolite, having a bulk silica to alumina ratio in the range of from 20 to 150 and being in its H⁺ form; and less than 10 wt. % of other components, all percentages being on the basis of total carrier; b) platinum in an amount in the range of from 0.001 to 0.1 wt. %, on the basis of total catalyst; and c) tin in an amount in the range of from 0.01 to 0.5 wt. %, on the basis of total catalyst. Said catalyst composition is described to be particularly suitable for use in ethylbenzene dealkylation. The working example in WO 2009/016143 A1 discloses a catalyst composition comprising ZSM-5 zeolite in combination with silica binder and platinum and tin as dopants therein.

CN 107398295 A describes catalysts for the catalytic purification of volatile organic compounds (VOC) and preparation thereof using co-mulling techniques wherein a supported base metal catalyst is mixed with the supported noble metal catalyst and calcined at 350-1000 C. The base metal includes one or more transition metals including Cr, Mn, Fe, Co, Ni, Zn and Cu and one or more rare earth metals including Y, La, Ce, Pr, Nd and Sm. The noble metal includes one or more of Pt, Pd, Ru, Ag, Au, Rh and Ir. The supports therein comprise one or more of an oxide support and a molecular sieve support. The oxide support includes Al₂O₃, SiO₂, TiO₂, ZrO₂ and other complexes. The molecular sieve support includes X, Y, M, A, CHA, MFI, Beta, and phosphorous aluminium molecular sieves. For example, Example 15 of CN 107398295 A discloses the preparation of a base metal catalyst of CeZrPrNd/Al₂O₃ and a noble metal catalyst comprising Pt, Pd, K and La on a ZSM-5 support. The prepared catalysts were then co-mulled by being added together in deionized water to form a pasty solid and then mechanically ground, fully mixed and calcined to produce a VOC catalyst. Such a co-mulled catalyst is very different in terms of its metals dispersion from a catalyst prepared wholly by impregnation techniques.

Some prior art documents describe dual-catalyst systems for use in the processing of BTX, in particular for the dealkylation of ethylbenzene and isomerisation of xylenes.

WO 2018/065474 A1 discloses a process for dealkylation of alkylaromatic compounds which process comprises contacting an alkylaromatic feedstock with i) a first catalyst comprising a) a carrier which comprises of from 20 to 70 wt. % of a refractory oxide binder and of from 30 to 80 wt. % of dealuminated ZSM-5 having a crystallite size of from 500 to 10,000 nm and a silica to alumina molar ratio (SAR) in the range of from 20 to 100; b) an amount of from 0.001 to 5 wt. % of one or more metals chosen from the group consisting of Groups 6, 9 and 10; and optionally c) a metal chosen from Group 14 in an amount up to 0.5 wt. %, and ii) a subsequent catalyst comprising a) a carrier which comprises of from 20 to 70 wt. % of a refractory oxide binder; of from 30 to 80 wt. % of ZSM-5 having a crystallite size of from 3 to 100 nm and a SAR in the range of from 20 to 200; b) an amount of from 0.001 to 5 wt. % of one or more metals chosen from the group consisting of Groups 6, 9 and 10; and optionally c) a metal chosen from Group 14 in an amount up to 0.5 wt. %, all percentages being on the basis of total catalyst.

However, notwithstanding the existing teaching described in the art to catalysts having efficacy in xylene isomerisation, as well having adequate catalytic performance, a further requirement for industrial application is that such catalysts should exhibit sufficient extrudate strength to avoid losses during downstream catalyst manufacturing processes, transport and to allow for loading of the catalyst into the reactor while preventing disintegration of the catalyst under commercial operation. Whilst catalysts known in the art, such as those comprising mixtures of zeolites, porous silica and silica sol, exhibit adequate catalytic performance, such catalysts can exhibit rather low extrudate strength. Accordingly, there is a continued need to develop catalysts exhibiting further improvements in the isomerisation of xylenes, and which, in particular, demonstrate advantageous xylene isomerisation in combination with high ethylbenzene conversion as well as sufficient strength to be applied in industrial xylene isomerisation processes.

### SUMMARY OF THE INVENTION

The present invention provides a process for the preparation of a catalyst composition, which process comprises:
forming a carrier from a mixture comprising a pentasil zeolite, one or more porous refractory oxide binders selected from alumina, amorphous silica-alumina, aluminum phosphate, magnesia, chromia, titania, boria and silica, and an aqueous solution of a zirconia precursor, and impregnating said carrier with metal dopants comprising one or more Group 10 metals selected from platinum, palladium and mixtures thereof in a total amount in the range of from 0.001 to 1 wt. % and, optionally, in the range of from 0.01 to 0.5 wt. % tin, based on the total weight of the catalyst composition.

The present invention further provides a catalyst composition made by said process.

Also provided is a xylene isomerisation process which comprises contacting an alkylaromatic feedstock with a catalyst composition prepared by the afore-mentioned process.

### DETAILED DESCRIPTION OF THE INVENTION

All weight amounts, as the term is used in relation with the catalyst composition or the catalyst preparation, are based on the basis of total catalyst and on dry amounts. Any water and other solvent present in the starting compounds are to be disregarded.

The primary particle diameter is measured by Scanning Electron Microscopy (SEM) with the average based on the number average.

Group 10 metals are as defined in the IUPAC Periodic Table of Elements dated 1 May 2013.

The weight amounts of metal are calculated as amount of metal on total weight of catalyst independent of the actual form of the metal.

The bulk or overall SAR can be determined by any one of a number of chemical analysis techniques. Such techniques include X-ray fluorescence, atomic adsorption, and inductive coupled plasma-atomic emission spectroscopy (ICP-AES). All techniques will provide substantially the same bulk ratio value. The molar silica-to-alumina ratio for use in the present invention is preferably determined by X-ray fluorescence.

In the present invention, it has been surprisingly found that the use of a specific zirconia precursor in the preparation of a carrier also comprising a pentasil zeolite and one or more porous refractory oxide binders and the use of said carrier in the preparation of a catalyst composition impregnated with one or more Group 10 metals selected from platinum, palladium and mixtures thereof and, optionally, tin as metal dopants, not only leads to a catalyst composition having increased strength and porosity, but which also demonstrates high activity and selectivity in xylene isomerisation processes.

The catalyst carrier in the catalyst composition of the present invention is formed from the mixture of a pentasil zeolite, one or more porous refractory oxide binders and an aqueous solution of a zirconia precursor.

Porous refractory oxide binders used in the catalyst composition of the present invention may be conveniently selected from one or more of alumina, amorphous silica-alumina, aluminum phosphate, magnesia, chromia, titania, boria and silica.

In a preferred embodiment of the present invention, the porous refractory oxide binder is selected from one or more of silica, amorphous silica-alumina and titania.

In a more preferred embodiment of the present invention, silica is used as the porous refractory oxide binder in the catalyst composition. The silica may be a naturally occurring silica or may be in the form of a gelatinous precipitate, sol or gel. The form of silica is not limited, and the silica may be in any of its various forms: crystalline silica, vitreous silica or amorphous silica. The term amorphous silica encompasses the wet process types, including precipitated silicas and silica gels, or pyrogenic or fumed silicas. Silica sols or colloidal silicas are non-settling dispersions of amorphous silicas in a liquid, usually water, typically stabilised by anions, cations, or non-ionic materials.

Conveniently, powder form silica has a B.E.T. surface area in the range of from 50 to 1000 m²/g; and a mean particle size in the range of from 2 nm to 200 μm, preferably in the range of from 2 to 100 μm, more preferably 2 to 60 μm, especially 2 to 10 μm as measured by ASTM C 690-1992 or ISO 8130-1.

Suitable binder materials that may be conveniently used are those available under the trade designation “Sipernat” from Evonik.

Preferably, the silica component is used as a pure silica and not as a component in another inorganic oxide. It is most preferred that, apart from the material derived from the zirconia precursor, the silica and indeed the carrier, is essentially free of any other inorganic oxide binder material, and especially is free of alumina. Preferably, at most only a maximum of 2 wt. % alumina, based on the total carrier, is present.

Pentasil zeolites are well known to the skilled person. ‘Pentasil’ is a term used to describe a class of shape-selective zeolites which are typically characterised by a silica to alumina ratio (SAR) of at least 12 and are constructed of five-membered rings (their framework being built up from 5-1 secondary building units). The pentasil zeolite utilised in the present invention preferably has a SAR in the range of from 20 to 150. The SAR is the bulk or overall silica/alumina ratio which may or may not be different to the framework SAR depending on any treatment to which the zeolite, either when free or in catalyst form, has been subjected.

Of the pentasil zeolites, the preferred zeolites are ZSM-5, ZSM-8, ZSM-11, ZSM-12, TON, e.g. ZSM-22, ZSM-23, ZSM-35, e.g. ferrierite, and ZSM-48, with those having the MFI configuration, and especially ZSM-5, being the most preferred. All of these zeolites are well known and documented in the literature, see for example the Database of Zeolite Structures: http://www.iza-structure.org/databases/ or Baerlocher et al “Atlas of zeolite framework types”, 5^{th} revised edition (2001), published on behalf of the Structure Commission of the International Zeolite Association, by Elsevier. Pentasil zeolites are reviewed in the Database at http://www.iza-structure.org/databases/Catalog/Pentasils.pdf.

Such zeolites can exist in various forms depending on the ion present at the cation sites in the zeolite structure. Generally the available forms contain an alkali metal ion, an alkaline earth metal ion, or a hydrogen or hydrogen precursor ion at the cation site. The zeolite may be used either in its template-free or its template-containing form.

The SAR of such zeolites is preferably at least 25, most preferably at least 30, and is preferably at most 100, most preferably at most 90, especially at most 85.

The zeolite starting material can exist in a number of particle size ranges. Suitably, the zeolite has a primary particle diameter in the range of from 5 nm to 10 μm. Useful catalysts have been prepared using a small particle size ZSM-5 having a primary particle diameter below 200 nm, preferably in the range of from 20 to 100 nm, more preferably in the range of from 25 to 80 nm. Suitable ZSM-5 materials can be prepared by procedures documented in the literature, for example in U.S. Pat. No. 3,702,886 A, in references provided in the Atlas, or Database, of Zeolite Structures, and in other literature references such as by Reding et al. in Microporous and Mesoporous Materials 57 (2003) 83 to 92, Yu et al. in Microporous and Mesoporous Materials 95 (2006) 234 to 240, and Iwayama et al. in U.S. Pat. No. 4,511,547 A.

Suitable grades of ZSM-5 zeolite include “CBV 3014E”, “CBV 8014”, and “CBV 3020E” zeolites, available commercially from Zeolyst International.

The zeolite is an important factor in the activity and selectivity properties shown by the catalyst composition of the invention. There is a balance between the activity and selectivity desired which may result in a different optimum zeolite content in the carrier depending on the zeolite used and the SAR of the zeolite used. Generally, a higher zeolite content may in some cases be advantageous to produce a higher activity from the catalyst composition, while a lower zeolite content may provide a higher selectivity.

While this balance may cause a different optimum depending on the specific conditions utilised in the xylene isomerisation process, generally it is preferred to maximise the amount of zeolite used in the catalyst carrier in a catalyst composition, since a higher amount of zeolite may improve the activity performance of catalyst composition, thereby allowing smaller reactors to be utilized and higher feedstock throughput. However, in the art it has been found that use of a higher zeolitic content can negatively affect the physical properties of the catalyst carrier such as lowering its strength, whilst tending to also allow more secondary reactions, thereby lowering selectivity.

In contrast, in the present invention, it has been surprisingly found that the use of zirconia derived from an aqueous solution of a zirconia precursor in the preparation of a catalyst composition also comprising a pentasil zeolite and one or more porous refractory oxide binders in combination with one or more Group 10 metals selected from platinum, palladium and mixtures thereof and, optionally, tin as metal dopants not only leads to a catalyst composition having increased strength and porosity, but also to a catalyst composition which demonstrates high activity and selectivity in xylene isomerisation processes.

As used herein, the carrier comprises a pentasil zeolite, one or more specified porous refractory oxide binders selected from alumina, amorphous silica-alumina, aluminum phosphate, magnesia, chromia, titania, boria and silica, and zirconia derived from an aqueous solution of a zirconia precursor. Preferably, the carrier consists of a pentasil zeolite, the one or more specified porous refractory oxide binders, and zirconia derived from an aqueous solution of a zirconia precursor.

In a preferred embodiment of the present invention, the carrier comprises at least 5 wt. %, more preferably in the range of from 5 to 50 wt. %, of a pentasil zeolite, at least 30 wt. %, more preferably in the range of from 30 to 90 wt. %, of the one or more porous refractory oxide binders selected from alumina, amorphous silica-alumina, aluminum phosphate, magnesia, chromia, titania, boria and silica, and at least 1 wt. %, more preferably in the range of from 1 to 30 wt. %, even more preferably in the range of from 3 to 30 wt. %, still more preferably in the range of from 3 to 25 wt. % and most preferably in the range of from 5 to 15 wt. %, of zirconia derived from the zirconia precursor, based on the weight of the catalyst composition on a dry basis.

A particularly preferred carrier comprises in the range of from 5 to 50 wt. % of a pentasil zeolite, in the range of from 30 to 90 wt. % of the one or more porous refractory oxide binders selected from alumina, amorphous silica-alumina, aluminum phosphate, magnesia, chromia, titania, boria and silica, and in the range of from 5 to 15 wt. %, of zirconia derived from the zirconia precursor, based on the weight of the catalyst composition on a dry basis.

In a more preferred embodiment of the present invention, wherein the porous refractory binder is selected from one or more of silica, amorphous silica-alumina and titania, the carrier comprises at least 5 wt. %, more preferably in the range of from 5 to 50 wt. %, of a pentasil zeolite, at least 30 wt. %, more preferably in the range of from 30 to 90 wt. %, of one or more porous refractory oxide binders selected from silica, amorphous silica-alumina and titania, and at least 1 wt. %, more preferably in the range of from 1 to 30 wt. %, even more preferably in the range of from 3 to 30 wt. %, still more preferably in the range of from 3 to 25 wt. % and most preferably in the range of from 5 to 15 wt. %, of zirconia derived from the zirconia precursor, based on the weight of the catalyst composition on a dry basis.

A particularly preferred carrier comprises in the range of from 5 to 50 wt. % of a pentasil zeolite, in the range of from 30 to 90 wt. % of one or more porous refractory oxide binders selected from silica, amorphous silica-alumina and titania, and in the range of from 5 to 15 wt. %, of zirconia derived from the zirconia precursor, based on the weight of the catalyst composition on a dry basis.

In a most preferred embodiment of the present invention, wherein the porous refractory oxide binder is silica, the carrier comprises in the range of from 5 to 50 wt. %, more preferably in the range of from 5 to 40 wt. %, even more preferably in the range of from 10 to 40 wt. % and most preferably in the range of from 10 to 25 wt. % of pentasil zeolite, in the range of from 30 to 90 wt. %, more preferably from 50 to 80 wt. % of silica, and at least 1 wt. %, more preferably in the range of from 1 to 30 wt. %, even more preferably in the range of from 3 to 30 wt. %, still more preferably in the range of from 3 to 25 wt. % and most preferably in the range of from 5 to 15 wt. %, of zirconia derived from the zirconia precursor, based on the weight of the catalyst composition on a dry basis.

A particularly preferred carrier comprises in the range of from 10 to 25 wt. % of a pentasil zeolite, in the range of from 50 to 80 wt. % of silica, and in the range of from 5 to 15 wt. %, of zirconia derived from the zirconia precursor, based on the weight of the catalyst composition on a dry basis.

A very suitable catalyst carrier for the present invention contains a pentasil zeolite, especially ZSM-5, having a SAR in the range of from 25 to 100, especially 30 to 85, in a preferred amount in the range of from 5 to 50 wt. %, more preferably in the range of from 5 to 40 wt. %, even more preferably in the range of from 10 to 40 wt. % and most preferably in the range of from 10 to 25 wt. %, based on the weight of the catalyst composition on a dry basis.

Preferably, the carrier in the catalyst composition of the present invention is formed only from the mixture of pentasil zeolite, the specified porous refractory oxide binder, in particular, silica, and an aqueous solution of a zirconia precursor. However, it is possible to include other components therein whilst still obtaining the benefits of the present invention. Such other components may be selected from other porous refractory oxide binder materials and other zeolites. Other porous refractory oxide binders may be alumina, and magnesia. Examples of other zeolites are 8, 10, or 12-membered ring zeolites, for example mordenite, and zeolite beta, and acidic mesoporous materials such as the MCM-series of zeolites, e.g. MCM-22 and MCM-41.

The carrier formed from the pentasil zeolite, the specified porous refractory oxide binder and an aqueous solution of a zirconia precursor is conveniently a shaped carrier and may be treated to enhance the activity of the zeolite component. In one embodiment of the present invention, the pentasil zeolite and/or the carrier undergoes treatment, such as dealumination, for example using ammonium hexafluorosilicate (AHS), coating or passivation and steaming.

In embodiments comprising carrier treatment, the treatment may be applied just once to the carrier formed from the pentasil zeolite, the specified porous refractory oxide binder and an aqueous solution of a zirconia precursor or may be applied two or more times.

For the avoidance of doubt, where a treatment has occurred that leaves silicon on the surface of the carrier, and where silica is used as the porous refractory oxide binder, this silicon content, which is usually only a small quantity, does not form part of the silica content of the carrier according to the invention.

The zirconia precursor for use in preparation of the catalyst composition of the present invention may be conveniently selected from ammonium zirconium carbonate, zirconium carbonate, zirconium acetate, zirconium citrate and zirconium oxalate. Ammonium zirconium carbonate, zirconium carbonate and zirconium acetate are preferred zirconia precursors for use in preparation of the catalyst composition of the present invention.

Commercially available zirconia precursors include those available under the trade designations “Bacote 20”, “Bacote M” and “Bacote XL” from Luxfer Mel Technologies.

The zirconia precursor is dissolved in a quantity of an aqueous solution. The aqueous solution may contain further compounds in addition to the zirconia precursor. For example, if the zirconia precursor is not soluble or is only partially soluble in water, then additional compounds may be present to solubilise the zirconia precursor. Such compounds may convert the zirconia precursor to forms that are more readily dissolved in the aqueous solution. Preferably, said zirconia precursor is dissolved in an aqueous alkaline solution. More preferably, said zirconia precursor is dissolved in an aqueous ammonia solution.

In shaped form, for example as extrudates, the carrier formed from the mixture of a pentasil zeolite, the specified porous refractory oxide binders and an aqueous solution of a zirconia precursor, generally has a B.E.T. surface area of at least 100 to 500 m²/g, preferably at least 130 m²/g, more preferably at least 150 m²/g; and a pore volume, by mercury intrusion, in the range of from 0.2 to 1.2 ml/g, preferably 0.4 to 1.1 ml/g, more preferably 0.7 to 1.1 ml/g. The flat plate crush strength generally is at least 80 N·cm⁻¹, preferably at least 100 N·cm⁻¹, and more preferably at least 140 N·cm⁻¹. It is generally, for example, of the order of 80 to 300 N·cm⁻¹, preferably 100 to 250 N·cm⁻¹, more preferably 140 to 200 N·cm⁻¹.

The catalyst composition of the invention also contains metal components in the form of one or more Group 10 metals selected from platinum, palladium and mixtures thereof. Platinum is particularly preferred as a Group 10 metal for use in the present invention.

Optionally, the catalyst composition of the invention may further comprise tin as an additional metal dopant.

The one or more Group 10 metals selected from platinum, palladium and mixtures thereof are present in a total amount in the range of from 0.001 to 1 wt. %, preferably in a total amount in the range of from 0.001 to 0.1 wt. %, more preferably in a total amount in the range of from 0.001 to 0.05 wt. %, and most preferably in a total amount in the range of from 0.005 to 0.05 wt. %, based on the total weight of the catalyst composition. The tin component is optionally present in an amount in the range of from 0.01 to 0.5 wt. %, preferably in an amount in the range of from 0.05 to 0.2 wt. %, based on the total weight of the catalyst composition.

With regard to the use of platinum as the Group 10 metal dopant, platinum may be present in an amount in the range of from 0.01 to 0.1, preferably 0.01 to 0.05 wt. %, based on the total weight of the catalyst composition. When used in conjunction with platinum, the tin component is most suitably present in an amount in the range of from 0.01 to 0.5, preferably in an amount in the range of from 0.05 to 0.2 wt. %, based on the total weight of the catalyst composition.

The catalyst composition of the invention has properties similar to that of the carrier formed from the mixture of a pentasil zeolite, porous refractory oxide binder and an aqueous solution of a zirconia precursor, in terms of B.E.T. surface area, pore volume and flat plate crush strength.

The catalyst composition of the present invention may be prepared using standard techniques for mixing the pentasil zeolite, porous refractory oxide binders such as silica, an aqueous solution of a zirconia precursor in an aqueous solution and optional other carrier components; shaping; compositing with the metal dopants; and any subsequent useful process steps such as drying, calcining, and reducing.

The shaping may be into any convenient form such as powders, extrudates, pills and granules. Preference is given to shaping by extrusion. To prepare extrudates, commonly the pentasil zeolite will be combined with the porous refractory oxide binders, preferably silica, an aqueous solution of a zirconia precursor and if necessary, a peptizing agent, and mixed to form a dough or thick paste. The peptizing agent may be any material that will change the pH of the mixture sufficiently to induce deagglomeration of the solid particles. Peptizing agents are well known and encompass organic and inorganic acids, such as nitric acid, and alkaline materials such as ammonia, ammonium hydroxide, alkali metal hydroxides, preferably sodium hydroxide and potassium hydroxide, alkali earth hydroxides and organic amines, e.g. methylamine and ethylamine. Ammonia is a preferred peptizing agent and may be provided in any suitable form, for example via an ammonia precursor. Examples of ammonia precursors are ammonium hydroxide and urea. It is also possible for the ammonia to be present as part of the silica component, particularly where a silica sol is used or the zirconia precursor if it is supplied in an ammonia solution, though additional ammonia may still be needed to impart the appropriate pH change. The amount of ammonia present during extrusion has been found to affect the pore structure of the extrudates which may provide advantageous properties. Suitably the amount of ammonia present during extrusion may be in the range of from 0 to 5 wt. % based on the total dry mixture, preferably 0 to 3 wt. %, more preferably 0 to 1.9 wt. %, on dry basis.

The metals emplacement onto the formed carrier may be by methods usual in the art. The metals can be deposited onto the carrier materials prior to shaping, but it is preferred to deposit them onto a shaped carrier.

Pore volume impregnation of the metals from a metal salt solution is a very suitable method of metals emplacement onto a shaped carrier. The metal salt solutions may have a pH in the range of from 1 to 12. The platinum salts that may conveniently be used are chloroplatinic acid, platinum nitrate and ammonium stabilised platinum salts. Examples of suitable tin salts utilised are stannous (II) chloride, stannic (IV) chloride, stannous oxalate, stannous sulphate, and stannous acetate. The metals may be impregnated onto the shaped carrier either sequentially or simultaneously. Where simultaneous impregnation is utilised the metal salts used must be compatible and not hinder the deposition of the metals.

The carrier formed from the mixture of a pentasil zeolite, porous refractory oxide binders and an aqueous solution of a zirconia precursor may be calcined before and/or after impregnation with metal dopants.

For example, after shaping of the carrier, and also after metals impregnation, the carrier/catalyst composition is suitably dried, and calcined. Drying temperatures are suitably 50 to 200° C.; drying times are suitably from 0.5 to 5 hours. Calcination temperatures are very suitably in the range of from 200 to 800° C., preferably 500 to 650° C. For calcination of the carrier, a relatively short time period is required, for example 0.5 to 3 hours.

Prior to use, it is necessary to ensure that the metals on the catalyst composition are in metallic (and not oxidic) form. Accordingly, it is useful to subject the composition to reducing conditions, which are, for example, heating in a reducing atmosphere, such as in hydrogen optionally diluted with an inert gas, or mixture of inert gases, such as nitrogen and carbon dioxide, at a temperature in the range of from 150 to 600° C. for at least 0.5 hours.

The catalyst composition of the invention finds particular use in the selective isomerisation of xylene.

The alkylaromatic feedstock for use in the process of the present invention most suitably originates indirectly from a reforming unit or naphtha pyrolysis unit. As described hereinbefore, BTX isolated from such units is subjected to distillation followed by extraction of para-xylene (p-xylene). The resultant para-xylene lean C8 aromatics stream may then by utilised as the alkylaromatic feedstock in the process of the present invention. Said feedstock may also be derived from the recycled effluent of a xylene isomerisation unit. Such feedstock usually comprises C₇ to C₈ hydrocarbons, and in particular one or more of o-xylene, m-xylene, p-xylene, toluene, and benzene in addition to ethylbenzene.

Preferably, the alkylaromatic feedstock comprises in the range of from 0.1 to 50 wt. % of ethylbenzene and in the range of from 20 to 99.9 wt. % of xylene, based on total amount of alkylaromatic feedstock.

Typically the xylenes will not be in a thermodynamic equilibrium, and the content of p-xylene will accordingly be lower than that of the other isomers.

The xylene isomerisation process may be carried out in a fixed bed system, a moving bed system, or a fluidised bed system. Such systems may be operated continuously or in batch fashion. Preference is given to continuous operation in a fixed bed system. The catalyst may be used in one reactor or in several separate reactors in series or operated in a swing system to ensure continuous operation during catalyst change-out.

The xylene isomerisation process of the present invention may be suitably carried out at a temperature in the range of from 300 to 500° C., a pressure in the range of from 0.1 to 50 bar (10 to 5,000 kPa), using a liquid weight hourly space velocity of in the range of from 0.5 to 25 h⁻¹. A partial pressure of hydrogen in the range of from 0.05 to 30 bar (5 to 3,000 kPa) is generally used. The feed to hydrogen molar ratio is in the range of from 0.2 to 100, generally from 1 to 10 mol/mol.

The present invention will now be illustrated by the following Examples.

### EXAMPLES

In the Examples and when mentioned elsewhere hereinabove, the following test methods are applicable: Flat plate crush strength: ASTM D 6175.

Water pore volume: the sample is dried at 300° C. for 1 hour and then weighed; water is added until the pores are filled such that the sample particles are wet but still free flowing; the sample is again weighed, and the amount of water absorbed per unit mass is calculated from the two weights.

### Example A (Comparative)

Demineralised water, ammonia and a colloidal silica binder (available from Nouryon under the trade designation “Levasil”) were added to a mix of small crystallite (primary particle diameters in the range of 3-100 nm) ZSM-5 zeolite (having a SAR of 30) and a porous silica powder (available from Evonik under the trade designation “Sipernat 50”). The weight ratio on a dry basis between ZSM-5 zeolite, porous silica powder and colloidal silica was 20:60:20. Demineralised water and ammonia were added to obtain an extrudable mix with a pH of 8-9.

The resulting material was mixed, mulled, extruded and calcined at 525° C. for 1 hour.

Afterwards, the extrudates were ion exchanged with 0.1 M NH₄Ac to remove any residual Na followed by calcination at 550° C. for 1 hour, and subsequently pore volume impregnated with hydrochloroplatinic acid and stannous chloride dissolved in 0.7 M hydrochloric acid to obtain 0.025 wt. % Pt and 0.1 wt. % Sn, based on the final weight of the catalyst composition.

After impregnation the catalyst was dried and calcined at 550° C. for 1 hour to obtain the final catalyst.

### Example B

Demineralised water, ammonia and an ammonium zirconium carbonate solution (available from Luxfer Mel Technologies under the trade designation “Bacote 20”) were added to a mix of small crystallite (primary particle diameters in the range of 3-100 nm) ZSM-5 zeolite (having a SAR of 30) and a porous silica powder (available from Evonik under the trade designation “Sipernat 50”). The weight ratio on a dry basis between ZSM-5 zeolite, porous silica powder and zirconia precursor was 20:75:5. Demineralised water and ammonia were added to obtain an extrudable mix with a pH of 8-9.

The resulting material was mixed, mulled, extruded and calcined at 525° C. for 1 hour.

Afterwards, the extrudates were ion exchanged with 0.3 M NH₄Ac to remove any residual Na followed by calcination at 550° C. for 1 hour, and subsequently pore volume impregnated with hydrochloroplatinic acid and stannous chloride dissolved in 0.7 M hydrochloric acid to obtain 0.025 wt. % Pt and 0.1 wt. % Sn, based on the final weight of the catalyst composition.

After impregnation the catalyst was dried and calcined at 550° C. for 1 hour to obtain the final catalyst.

### Example C

Demineralised water, ammonia and an ammonium zirconium carbonate solution (available from Luxfer Mel Technologies under the trade designation “Bacote 20”) were added to a mix of small crystallite (primary particle diameters in the range of 3-100 nm) ZSM-5 zeolite (having a SAR of 30) and a porous silica powder (available from Evonik under the trade designation “Sipernat 50”). The weight ratio on a dry basis between ZSM-5 zeolite, porous silica powder and zirconia precursor was 20:50:30. Demineralised water and ammonia were added to obtain an extrudable mix with a pH of 8-9.

The resulting material was mixed, mulled, extruded and calcined at 525° C. for 1 hour.

Afterwards, the extrudates were ion exchanged with 0.3 M NH₄Ac to remove any residual Na followed by calcination at 550° C. for 1 hour, and subsequently pore volume impregnated with hydrochloroplatinic acid and stannous chloride dissolved in 0.7 M hydrochloric acid to obtain 0.025 wt. % Pt and 0.1 wt. % Sn, based on the final weight of the catalyst composition.

After impregnation the catalyst was dried and calcined at 550° C. for 1 hour to obtain the final catalyst.

### Example D (Comparative)

Demineralised water and ammonia were added to a mix of small crystallite (primary particle diameters in the range of 3-100 nm) ZSM-5 zeolite (having a SAR of 30), porous zirconia powder (available from Daiichi Kigenso Kagaku Kogyo Co., Ltd.) and a porous silica powder (available from Evonik under the trade designation “Sipernat 50”). The weight ratio on a dry basis between ZSM-5 zeolite, porous silica powder and porous zirconia powder was 20:75:5. Demineralised water and ammonia were added to obtain an extrudable mix with a pH of 8-9.

The resulting material was mixed, mulled, extruded and calcined at 525° C. for 1 hour.

Afterwards, the extrudates were ion exchanged with 0.3 M NH₄Ac to remove any residual Na followed by calcination at 550° C. for 1 hour, and subsequently pore volume impregnated with hydrochloroplatinic acid and stannous chloride dissolved in 0.7 M hydrochloric acid to obtain 0.025 wt. % Pt and 0.1 wt. % Sn, based on the final weight of the catalyst composition.

After impregnation the catalyst was dried and calcined at 550° C. for 1 hour to obtain the final catalyst.

### Example E (Comparative)

Demineralised water and ammonia were added to a mix of small crystallite (primary particle diameters in the range of 3-100 nm) ZSM-5 zeolite (having a SAR of 30), porous zirconia powder (available from Daiichi Kigenso Kagaku Kogyo Co., Ltd.) and a porous silica powder (available from Evonik under the trade designation “Sipernat 50”). The weight ratio on a dry basis between ZSM-5 zeolite, porous silica powder and porous zirconia powder was 20:50:30. Demineralised water and ammonia were added to obtain an extrudable mix with a pH of 8-9.

The resulting material was mixed, mulled, extruded and calcined at 525° C. for 1 hour.

Afterwards, the extrudates were ion exchanged with 0.3 M NH₄Ac to remove any residual Na followed by calcination at 550° C. for 1 hour, and subsequently pore volume impregnated with hydrochloroplatinic acid and stannous chloride dissolved in 0.7 M hydrochloric acid to obtain 0.025 wt. % Pt and 0.1 wt. % Sn, based on the final weight of the catalyst composition.

After impregnation the catalyst was dried and calcined at 550° C. for 1 hour to obtain the final catalyst.

### Example F (Comparative)

Demineralised water, ammonia and an ammonium zirconium carbonate solution (available from Luxfer Mel Technologies under the trade designation “Bacote 20”) were added to a mix of small crystallite (primary particle diameters in the range of 3-100 nm) ZSM-5 zeolite (having a SAR of 30) and a porous zirconia powder (available from Daiichi Kigenso Kagaku Kogyo Co., Ltd.). The weight ratio on a dry basis between ZSM-5 zeolite, porous zirconia powder and zirconia precursor was 20:75:5. Demineralised water and ammonia were added to obtain an extrudable mix with a pH of 8-9.

The resulting material was mixed, mulled, extruded and calcined at 525° C. for 1 hour.

Afterwards, the extrudates were ion exchanged with 0.3 M NH₄Ac to remove any residual Na followed by calcination at 550° C. for 1 hour, and subsequently pore volume impregnated with hydrochloroplatinic acid and stannous chloride dissolved in 0.7 M hydrochloric acid to obtain 0.025 wt. % Pt and 0.1 wt. % Sn, based on the final weight of the catalyst composition.

After impregnation the catalyst was dried and calcined at 550° C. for 1 hour to obtain the final catalyst.

### Testing of Catalysts

Testing was conducted of the catalysts shown in Table 1 under catalytic testing conditions that mimics typical industrial application conditions for the combined system of ethylbenzene dealkylation and xylene isomerisation.

TABLE 1

| | ZSM-5 | Binder in Carrier | | | | | |
|---|---|---|---|---|---|---|---|
| | Zeolite in | (wt. %) | | | | Carrier Dopants | |
| | Carrier* | Porous Silica | Colloidal | Zirconia | Porous Zirconia | (wt. %) | |
| Example | (wt. %) | Powder | Silica | Precursor | Powder | Pt | Sn |
| A (Comp.) | 20 | 60 | 20 | — | — | 0.025 | 0.1 |
| B | 20 | 75 | — | 5 | — | 0.025 | 0.1 |
| C | 20 | 50 | — | 30 | — | 0.025 | 0.1 |
| D (Comp.) | 20 | 75 | — | — | 5 | 0.025 | 0.1 |
| E (Comp.) | 20 | 50 | — | — | 30 | 0.025 | 0.1 |
| F (Comp.) | 20 | — | — | 5 | 75 | 0.025 | 0.1 |

*The same zeolite was used in the working and comparative examples.

The activity test used a feed having the composition summarised in Table 2.

TABLE 2
Composition of the feed used in the activity testing

| | (wt. % in feed |
|---|---|
| Component | composition) |
| Ethylbenzene (EB) | 7.5 |
| p-Xylene (pX) | 1.7 |
| o-Xylene (oX) | 26.3 |
| m-Xylene (mX) | 63.8 |
| C₇-C₉-paraffins | 0.7 |
| C₉₊ aromatics | 0.00 |
| Total | 100.00 |

Other Feed Characteristics

| | |
|---|---|
| C₈ aromatics sum (wt. %) | 99.3 |
| EB in C8 aromatics feed (wt. %) | 7.6 |
| pX (wt. %) in xylenes in feed | 1.9 |
| oX (wt. %) in xylenes in feed | 28.6 |
| mX (wt. %) in xylenes in feed | 69.5 |

The activity test was performed in a fixed bed unit with online GC analysis once the catalyst was in its reduced state, which was achieved by exposing the dried and calcined catalyst to atmospheric hydrogen (>99% purity) at 450° C. for 1 hour.

After reduction, the reactor was cooled down to 380° C., pressurised to 0.8 MPa and the feed was introduced at a weight hourly space velocity of 25 g feed/g catalyst/hour and a hydrogen to feed ratio of 2.4 mol·mol⁻¹. Subsequently, the temperature is increased to 450° C. and the hydrogen to feed ratio to 0.5 mol·mol⁻¹. This step contributes to enhanced catalyst aging, and therefore allows comparison of the catalytic performance at stable operation. After 24 hours the conditions were switched to the actual operating conditions.

In the present case, a weight hourly space velocity of 25 g feed/g catalyst/hour, hydrogen to feed ratio of 2.4 mol·mol⁻¹ and a total system pressure of 0.8 MPa was used. The temperature was varied between 340 and 390° C.

### Results

Ethylbenzene conversion (EB conversion) is the weight percent of ethylbenzene converted by the catalyst into benzene and ethylene, or other molecules. It is defined as wt. % ethylbenzene in feed minus wt. % ethylbenzene in product divided by wt. % ethylbenzene in feed times 100%.

PX/X is the percentage of para-xylene (pX) in xylenes (pX, meta-xylene (mX) and ortho-xylene (oX)) in the effluent.

The performance characteristics including the products obtained are shown in Table 3 below, wherein comparisons are made to determine the temperature required to achieve at a PX/X of 24.0%.

In addition, the performance characteristics including the products obtained are shown in Table 4 below, wherein comparisons are made at 365° C. The PX/X equilibrium concentration at 365° C. is assumed to be 24.2%.

The formation of C₈ aromatic components, such as trimethylbenzene (TMB) and methylethylbenzene (MEB) and C₁₀ or larger aromatic components are unwanted as they form at the expense of xylene molecules (xylene loss).

TABLE 3

| | Temp. (° C.) | | | | Porosity | |
|---|---|---|---|---|---|---|
| | (@ PX/X | TMB in | MEB in | | (water pore | |
| | (%) = | product, | product, | Xylene | volume, | Strength |
| Example | 24.0%) | wt. % | wt. % | losses | ml/g) | (N/cm) |
| A (Comp.) | 387 | 0.36 | 0.06 | 0.79 | 0.87 | 147 |
| B | 381 | 0.20 | 0.06 | 0.42 | 1.00 | 151 |
| D (Comp.) | 341 | 0.29 | 0.09 | 0.63 | 1.09 | 68 |
| F (Comp.) | 381 | 1.64 | 0.29 | 3.77 | 0.53 | 46 |

TABLE 4

| | | | | | Porosity | |
|---|---|---|---|---|---|---|
| | PX/X (%) | TMB in | MEB in | | (water pore | |
| | (@ temp. = | product, | product, | Xylene | volume, | Strength |
| Example | 365° C.) | wt. % | wt. % | losses | ml/g) | (N/cm) |
| A (Comp.) | 23.90 | 0.21 | 0.06 | 0.44 | 0.87 | 147 |
| B | 23.87 | 0.13 | 0.05 | 0.26 | 1.00 | 151 |
| C | 24.02 | 0.61 | 0.13 | 1.38 | 0.68 | 105 |
| D (Comp.) | 24.11 | 0.44 | 0.08 | 0.99 | 1.09 | 68 |
| E (Comp.) | 24.07 | 0.68 | 0.12 | 1.55 | 0.77 | 64 |
| F (Comp.) | 23.93 | 1.24 | 0.29 | 2.87 | 0.53 | 46 |

### Discussion of Results

The above experimental results show that the use of zirconia derived from an aqueous solution of a zirconia precursor in combination with a pentasil zeolite and silica binder in a carrier surprisingly results in a catalyst having not only advantageous catalytic performance in xylene isomerisation, but also having improved strength.

Comparison of the results of Example B vis-à-vis comparative Example A demonstrates that the use of 5 percent on a dry basis of an ammonium-stabilized zirconium carbonate solution in the preparation of a xylene isomerisation catalyst of the present invention resulted in an increased carrier porosity while achieving at the same time a slightly higher crush strength. Furthermore, the use of the afore-mentioned zirconium carbonate solution during synthesis not only increased the catalytic activity for xylene isomerisation slightly (Table 3), but also significantly reduced the formation of unwanted by-products such as TMB, MEB and C₁₀₊ components (Tables 3 and 4).

Comparison of the results of Example B vis-à-vis comparative Example D in Tables 3 and 4 demonstrate that the beneficial results obtained in Example B are not demonstrated when an alternative zirconia source is employed during catalyst manufacture (in comparative Example D). Thus, it is apparent that the use of 5 percent on a dry basis of an ammonium-stabilized zirconium carbonate solution in the preparation of a xylene isomerisation catalyst of the present invention resulted in a significantly higher crush strength and also reduced formation of unwanted by-products such as TMB, MEB and C₁₀₊ components in xylene isomerisation.

Example C utilised an increased amount of the ammonium-stabilized zirconium solution to 30 percent on a dry basis.

Comparing the results of Example C with comparative Example E (which utilised the same amount of zirconia, but from an alternative source) in Table 4, it is apparent that Example C not only exhibited advantageous PX/X selectivity, but also showed reduced TMB make and xylene losses and higher strength than the catalyst of comparative Example E.

Comparative Example F utilised 5 percent on a dry basis of an ammonium-stabilized zirconium carbonate solution in combination with zeolite and zirconium powder. Comparing the results of comparative Example F with Example B (in Tables 3 and 4), it is observed that the amount of unwanted by-products (i.e. TMB and MEB) and xylene losses are greatly increased. Furthermore, comparative Example F showed poor crush strength vis-à-vis Example B.

In summary, it has been surprisingly found in the present invention that the use of an ammonium-stabilized zirconium solution in combination with zeolite and certain refractory oxide binders leads to good crush strength together with good xylene isomerization activity and very low xylene losses.

1. A process for the preparation of a catalyst composition, which process comprises:
forming a carrier from a mixture comprising a pentasil zeolite, one or more porous refractory oxide binders selected from alumina, amorphous silica-alumina, aluminum phosphate, magnesia, chromia, titania, boria and silica, and an aqueous solution of a zirconia precursor, and impregnating said carrier with metal dopants comprising one or more Group 10 metals selected from platinum, palladium and mixtures thereof in a total amount in the range of from 0.001 to 1 wt. % and, optionally, in the range of from 0.01 to 0.5 wt. % tin, based on the total weight of the catalyst composition.

2. Process according to claim 1, wherein said carrier comprises at least 5 wt. % of a pentasil zeolite, at least 30 wt. % of the one or more porous refractory oxide binders, and at least 1 wt. of zirconia derived from the zirconia precursor, based on the weight of the catalyst composition on a dry basis.

3. Process according to claim 1, wherein the one or more porous refractory oxide binders are selected from one or more of silica, amorphous silica-alumina and titania.

4. Process according to a claim 1, wherein the one or more porous refractory oxide binders are silica.

5. Process according to claim 1, wherein the pentasil zeolite is one having an MFI configuration.

6. Process according to claim 1, wherein the pentasil zeolite is ZSM-5.

7. Process according to claim 1, wherein the zirconia precursor is dissolved in an aqueous alkaline solution.

8. Process according to claim 1, wherein the zirconia precursor is dissolved in an aqueous ammonia solution.

9. Process according to claim 1, wherein the zirconia precursor is selected from ammonium zirconium carbonate, zirconium carbonate, zirconium acetate, zirconium citrate and zirconium oxalate.

10. Process according to claim 1, wherein the Group 10 metal is platinum.

11. Process according to claim 1, wherein the carrier is calcined before and/or after impregnation with metal dopants.

12. A catalyst composition prepared by the process of claim 1.

13. A xylene isomerisation process which comprises contacting an alkylaromatic feedstock with a catalyst composition prepared by a process as claimed in claim 1 or a catalyst composition as claimed in claim 12.

14. Process according to claim 13, wherein the alkylaromatic feedstock comprises in the range of from 0.1 to 50 wt. % of ethylbenzene and in the range of from 20 to 99.9 wt. % of xylene, based on total amount of alkylaromatic feedstock.
